(19) United States
(12) Patent Application Publication
(10) Pub. No.: US 2026/0242675 A1
(43) Pub. Date: Aug. 20, 2026

(54) METHOD FOR LIQUID-LIQUID EXTRACTION OF AROMATICS WITH EXTRACT RECYCLES STREAMS

(71) Applicant: IFP ENERGIES NOUVELLES, Rueil-Malmaison (FR)

(72) Inventors: Pierre-Alain BREUIL, Rueil-Malmaison Cedex (FR); Sophie COUDERC, Rueil-Malmaison Cedex (FR); Rejane DASTILLUNG, Rueil-Malmaison Cedex (FR); Jeremy GAZARIAN, Rueil-Malmaison Cedex (FR)

(73) Assignee: IFP ENERGIES NOUVELLES, Rueil-Malmaison (FR)

(21) Appl. No.: 18/852,033

(22) PCT Filed: Mar. 14, 2023
(86) PCT No.: PCT/EP2023/056439

§ 371 Date: Sep. 27, 2024

(30) Foreign Application Priority Data
Mar. 28, 2022 (FR) FR2202765

(51) Int. Cl. C10G 21/06; C10G 21/28; C10G 21/30

(52) U.S. Cl. CPC C10G 21/06; C10G 21/28; C10G 21/30; C10G 2300/1096; C10G 2300/201; C10G 2300/4081; C10G 2400/30

(57) ABSTRACT

The present invention relates to a process and to a device for the separation of aromatic compounds from a feedstock (1) comprising a mixture of aromatic and non-aromatic compounds, in which aromatics are extracted from the feedstock in a liquid-liquid extractor (T1) fed with a solvent stream (2) in order to produce at least an extract (4) concentrated in aromatic compounds, with respect to the feedstock, and a raffinate (3) concentrated in non-aromatic compounds, with respect to the composition of the feedstock; and in which an upper part (Z1) and a lower part (Z2) of the liquid-liquid extractor (T1) are fed respectively with a first fraction (51) and a second fraction (52) of extract recycled material (5), the upper part and the lower part being defined with respect to the position of the feed point of the feedstock in the liquid-liquid extractor.

### TECHNICAL FIELD

The field of the invention relates to the separation of aromatic compounds, such as benzene, toluene, xylenes (BTX), but also aromatic compounds having 9, 10 or 11 carbon atoms (A9, A10, A11), for the petrochemical industry originating from a feedstock comprising a mixture of aromatic and non-aromatic compounds, such as a petrol feedstock.

### PRIOR ART

With reference to the work “Procédé séparation” [Separation Process]” of J.-P. Wauquiez (Editions Technip, 1998, pp. 438-444) and to the field of the invention described above, the process for the extraction of BTX aromatics comprises three main operations, which are the extraction, the purification of the aromatics and the regeneration of the solvent. The prior art relates to the process for the extraction of the aromatics comprising a column for the liquid-liquid extraction of the aromatics, an extractive distillation column and a column for regeneration of the solvent. The feedstock comprising a mixture of aromatics and non-aromatics is sent into the middle of the extraction column, the solvent is sent to the top of the column and the extraction is carried out by bringing the feedstock and the solvent into contact over the top section of the extraction column. Within the same column, the solvent charged with aromatics enters a “backwash” zone where said solvent is brought into contact with a recycling of a mixture of light aromatics/non-aromatics originating from the top of the extractive distillation column, making it possible to partially purify the aromatics in order to produce an extract. The extract is sent to an extractive distillation column for purification by means of a boil-up rate adjusted in order for the aromatics at the column bottom to be at predetermined specifications of content of non-aromatics. The purified extract is sent into a column for regeneration by azeotropic distillation which makes it possible to separate the aromatics from the solvent.

### SUMMARY OF THE INVENTION

In the context described above, a first object of the present description is to provide a process for the separation of a hydrocarbon feedstock for the production of aromatics with an improved yield, which are in particular compatible with injection into an aromatics complex.

According to a first aspect, the abovementioned objects, and also other advantages, are obtained by a process for the separation of aromatic compounds from a feedstock comprising a mixture of aromatic and non-aromatic compounds, the process comprising the following stages:
extracting the aromatics from the feedstock by means of a liquid-liquid extractor fed with a solvent stream, in order to produce at least an extract concentrated in aromatic compounds, with respect to the composition of the feedstock, and a raffinate concentrated in non-aromatic compounds, with respect to the composition of the feedstock;
feeding an upper part and a lower part of the liquid-liquid extractor with respectively a first fraction and a second fraction of extract recycled material, the upper part and the lower part being defined with respect to the position of the feed point of the feedstock in the liquid-liquid extractor.

According to one or more embodiments, the solvent stream 2 feeds the top of the upper part Z1 of the liquid-liquid extractor T1.

According to one or more embodiments, the first fraction feeds said upper part at a point arranged at a position of between 0.05×L1 and 0.95×L1, preferably between 0.2×L1 and 0.9×L1 and preferably between 0.3×L1 and 0.8×L1, L1 being the length from the top of the liquid-liquid extractor T1 to the position of the feed point of the feedstock.

According to one or more embodiments, the first fraction feeds said upper part below the feed point of the solvent stream.

According to one or more embodiments, the second fraction feeds said lower part at a point arranged at a position of between 0×L2 and 0.3×L2, preferably between 0×L2 and 0.2×L2 and preferably between 0×L2 and 0.1×L2, L2 being the length from the position of the bottom of the liquid-liquid extractor T1 to the feed point of the feedstock.

According to one or more embodiments, the ratio by weight of the first fraction to the second fraction is between 0.01 and 0.99, preferably between 0.1 and 0.95, preferably between 0.15 and 0.85, and preferably between 0.2 and 0.8.

According to one or more embodiments, the ratio by weight of the solvent to the feedstock is between 0.1 and 50, preferably between 0.5 and 20, preferably between 1 and 9, preferably between 3 and 8.

According to one or more embodiments, the ratio by weight of the extract recycled material to the feedstock is between 0.05 and 10, preferably between 0.1 and 8, preferably between 0.2 and 5, preferably between 0.3 and 2.

According to one or more embodiments, the feedstock contains aromatic and non-aromatic compounds comprising from 6 to 11 carbon atoms.

According to one or more embodiments, the liquid-liquid extractor is operated at at least one of the following operating conditions: a pressure of between 0.05 MPa and 3 MPa, preferably between 0.1 MPa and 2 MPa, preferably between 0.2 MPa and 1.5 MPa, preferably between 0.3 MPa and 1 MPa; a temperature of between 10° C. and 150° C., preferably between 15° C. and 130° C., preferably between 30° C. and 120° C., preferably between 40° C. and 110° C.

According to one or more embodiments, the process comprises the following stages:
stripping of the extract by means of an extract stripping section in order to separate a gas stream comprising non-aromatic compounds and a purified extract;
separation of the aromatics from the purified extract by means of an aromatic recovery tower in order to separate the solvent stream and top vapours comprising an aromatic stream.

According to one or more embodiments, the process comprises at least one of the following stages:
feeding the liquid-liquid extractor with a solvent stream comprising a solvent chosen from the list consisting of ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these;
phase separation of the gas stream in a condenser-settler in order to separate the extract recycled material and an aqueous phase;
scrubbing the raffinate with water by means of a water scrubbing tower fed with water in order to produce a non-aromatic stream and aqueous scrubbing liquor;
regeneration of at least a portion of the solvent stream by means of a section for regeneration of the solvent by vacuum steam stripping;
condensation of the top vapours in a condenser-settler in order to produce the aromatic stream.

According to one or more embodiments, the process comprises at least one of the following stages:
feeding the liquid-liquid extractor with a stream of solvent chosen from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these;
stripping the hydrocarbon compounds present in the aqueous scrubbing liquor and/or in the aqueous phase by means of a water stripping section in order to produce water;
feeding the aromatic recovery tower with steam in order to produce top vapours additionally comprising steam, and condensation of the top vapours in the condenser-settler in order to produce the aromatic stream and water separated by settling.

According to one or more embodiments, the process comprises at least one of the following stages:
feeding the liquid-liquid extractor with a solvent stream comprising sulfolane and water;
recycling the water produced by the water stripping section to the aromatic recovery tower.

According to a second aspect, the abovementioned objects, and also other advantages, are obtained by a device for the separation of aromatic compounds from a feedstock comprising a mixture of aromatic and non-aromatic compounds, the device comprising a liquid-liquid extractor suitable for extracting aromatics from the feedstock with a solvent stream and producing at least an extract concentrated in aromatic compounds, with respect to the feedstock, and a raffinate concentrated in non-aromatic compounds, with respect to the composition of the feedstock, the liquid-liquid extractor comprising:
a feed point for the feedstock;
a feed point for the solvent stream;
a feed point for a first fraction of extract recycled material arranged in an upper part of the liquid-liquid extractor;
a feed point for a second fraction of extract recycled material arranged in a lower part of the liquid-liquid extractor, the upper part and the lower part being defined with respect to the position of the feed point of the feedstock in the liquid-liquid extractor.

Embodiments according to the abovementioned aspects and also other characteristics and advantages will become apparent on reading the description which follows, given solely by way of illustration and without limitation, and with reference to the following drawings.

### LIST OF THE FIGURES

FIG. 1 diagrammatically shows a separation process according to the invention.

FIG. 2 diagrammatically shows the operating principle of the liquid-liquid extractor according to the invention.

FIG. 3 diagrammatically shows a separation process according to the prior art.

### DESCRIPTION OF THE EMBODIMENTS

Embodiments of the invention will now be described in detail. In the following detailed description, many specific details are presented in order to provide a deeper understanding of the invention. However, it will be apparent to a person skilled in the art that the invention can be implemented without these specific details. In other cases, well-known characteristics have not been described in detail in order to avoid unnecessarily complicating the description.

In the present patent application, the term “to comprise” is synonymous with (means the same thing as) “to include” and “to contain”, and is inclusive or open and does not exclude other elements not stated. It is understood that the term “to comprise” includes the exclusive and closed term “to consist”. The term “based on” is synonymous with “comprises at least 50% by weight of”. In addition, in the present description, the terms “essentially” or “substantially” correspond to an approximation of ±5%, preferably of ±1%, very preferably of ±0.5%. For example, an effluent essentially comprising or being constituted of the compounds A corresponds to an effluent comprising at least 95% by weight of compounds A. In the present description, the term “stream concentrated in a compound” corresponds, for example, to a stream comprising at least 80% by weight, preferably at least 90% by weight, very preferably at least 95% by weight, of said compound.

The present invention relates to the separation of aromatic compounds, and in particular of BTX compounds, but also aromatic compounds having 9 or 10, indeed even 11, carbon atoms (A9, A10, A11), for the petrochemical industry originating, for example, from a petrol feedstock, such as a catalytic cracking petrol. It relates in particular to an improved process and device for the separation of the aromatic compounds of 6 to 11 carbon atoms from a feedstock comprising a mixture of aromatic and non-aromatic compounds, such as a petrol feedstock.

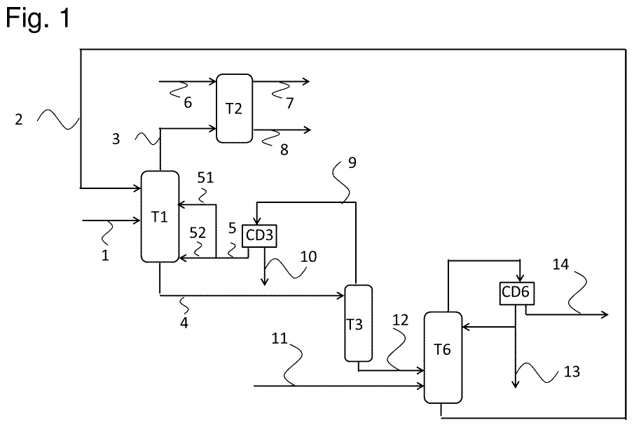

Specifically with reference to FIG. 1, the process according to the invention makes it possible to treat the feedstock 1 with a solvent stream 2 in a liquid-liquid extractor T1 in order to produce a stream concentrated (i.e. enriched) in non-aromatic compounds (raffinate 3) and a stream concentrated in aromatic compounds (extract 4), in which process both an upper part Z1 and a lower part Z2 of the liquid-liquid extractor T1 are fed with an extract recycled material 5. Advantageously, the aromatic separation process/device according to the invention makes it possible to obtain an improved yield for recovery of the aromatics while guaranteeing a high purity, in particular BTX purity. In addition, the raffinate 3 (which is optionally scrubbed) can be sent to a thermal cracking stage/unit or else to a catalytic reforming stage/unit, for example in order to increase the production of aromatics.

### The Feedstock

The separation process according to the invention makes it possible to treat a feedstock 1 comprising a mixture of aromatic and non-aromatic compounds. Preferably, the feedstock 1 is hydrotreated and/or hydrogenated. According to one or more embodiments, the feedstock 1 is an optionally hydrotreated and/or hydrogenated petrol feedstock.

According to one or more embodiments, the feedstock 1 is a C5+ cut, i.e. containing compounds having 5 and more carbon atoms. According to one or more embodiments, the feedstock 1 is a C5-C10 or C5-C11 cut, i.e. a cut containing compounds comprising from 5 to 10 or from 5 to 11 carbon atoms. According to one or more embodiments, the feedstock 1 is a C6-C10 or C6-C11 cut, i.e. a cut containing compounds comprising from 6 to 10 or from 6 to 11 carbon atoms.

According to one or more embodiments, the feedstock 1 comprises at least 20% by weight, preferably at least 30% by weight, very preferably at least 40% by weight (e.g. at least 50% by weight), of aromatic compounds of 6 to 11 carbon atoms, with respect to the total weight of the feedstock.

According to one or more embodiments, the feedstock 1 comprises at least 20% by weight, preferably at least 30% by weight, very preferably at least 40% by weight (e.g. at least 50% by weight), of monoaromatic compounds of 6 to 11 carbon atoms, with respect to the total weight of the feedstock.

According to one or more embodiments, the aromatic compounds of the feedstock 1 are monoaromatic compounds at at least 95% by weight, preferably at at least 98% by weight, very preferably at at least 99% by weight.

According to one embodiment of the invention, the feedstock 1 comprises less than 50 ppm by weight of sulfur, preferably less than 10 ppm by weight of sulfur and very preferably less than 1 ppm by weight of sulfur.

According to one embodiment of the invention, the feedstock 1 comprises less than 100 ppm by weight of nitrogen, preferably less than 10 ppm by weight of nitrogen and very preferably less than 1 ppm by weight of nitrogen.

According to one embodiment of the invention, the feedstock 1 comprises less than 0.1% by weight of diolefins, preferably less than 0.05% by weight of diolefins and very preferably less than 0.01% by weight of diolefins.

According to one embodiment of the invention, the feedstock 1 comprises less than 0.1% by weight of olefins, preferably less than 0.05% by weight of olefins and very preferably less than 0.01% by weight of olefins.

According to one embodiment of the invention, the feedstock 1 exhibits a content of less than or equal to 5000 ppm by weight, preferably of less than or equal to 4500 ppm by weight and very preferably of less than or equal to 3000 ppm by weight, of compounds having a boiling point of greater than 217° C., such as naphthalene.

According to one embodiment of the invention, the feedstock 1 is devoid of the following compounds: H₂, H₂S, light gas, such as ethane, propane and butane. According to one embodiment of the invention, the removal of these compounds from the feedstock 1 is carried out in a fractionation column.

According to one embodiment, said feedstock 1 is at least in part a petrol cut resulting from a fluidized bed catalytic cracking unit (FCC (for Fluid Catalytic Cracking) unit), the petrol cut preferably having been selectively hydrogenated in order to convert diolefins into olefins, then fractionated in order to obtain a C5-C10, C5-C11, C6-C10 or C6-C11 cut, then hydrogenated in order to saturate the olefin compounds. According to one embodiment of the invention, the feedstock 1 results from the hydrogenation of a pyrolysis gasoline (PyGas) as a mixture with a petrol cut resulting from an FCC unit.

### Extraction

The extraction of the aromatics in the present invention makes it possible to treat the feedstock 1 in order to recover, on the one hand, a stream concentrated in non-aromatic compounds, referred to as raffinate 3, and, on the other hand, a stream concentrated in aromatics, referred to as extract 4, with respect to the composition of the feedstock 1.

According to one or more embodiments, with reference to FIG. 1, the extraction of the aromatics is a liquid-liquid extraction of the aromatics.

According to one or more embodiments, the liquid-liquid extraction of the aromatics comprises the following stages:
liquid-liquid extraction by means of a liquid-liquid extractor T1 fed with a solvent stream 2 in order to separate the raffinate 3 and the extract 4,
water scrubbing of the raffinate 3 with a water scrubbing tower T2 (optional stage),
stripping of the water used in the scrubbing tower T2 with a water stripping section (optional stage),
stripping of the extract 4 with an extract stripping section T3,
separation of the aromatics with a recovery tower T6 in order to separate an aromatic stream 13 and the solvent stream 2, and
regeneration of the solvent with a solvent regeneration section (optional stage).

For the sake of simplicity, the optional water stripping and solvent regeneration sections are not represented in FIG. 1.

### Liquid-Liquid Extractor T1

The liquid-liquid extraction makes possible the separation of the aromatics from the non-aromatic compounds, such as paraffins and naphthenes, of the feedstock 1.

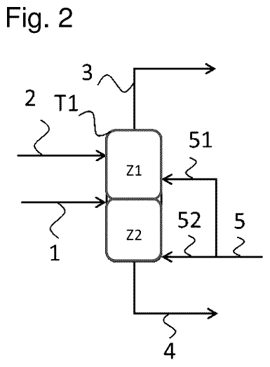

With reference to FIG. 2, feedstock 1 is fed substantially at an intermediate point in the liquid-liquid extractor T1 (e.g., in the middle of the extractor).

According to one or more embodiments, the intermediate point is a point located between the top and the bottom of the liquid-liquid extractor T1. According to one or more embodiments, the intermediate point corresponds to a point arranged at a position between 0.1×L and 0.9×L, more preferably between 0.2×L and 0.8×L, such as between 0.3×L and 0.7×L or between 0.4×L and 0.6×L, L being the length (starting) from the top (down) to the bottom of the liquid-liquid extractor T1.

Advantageously, the liquid-liquid extractor T1 can thus be divided into two parts. The upper part Z1 (with respect to the intermediate point for feeding with the feedstock 1) makes it possible to control the yield of aromatics by liquid-liquid extraction (in particular with solvent). The lower part Z2 of the liquid-liquid extractor T1 makes possible a first purification of the aromatics, in particular by means of a stream of extract recycled material 5.

According to one or more embodiments, the solvent stream 2 feeds the upper part Z1 of the liquid-liquid extractor T1. According to one or more embodiments, the solvent stream 2 feeds the top of the upper part Z1 of the liquid-liquid extractor T1.

According to one or more embodiments, the solvent comprises a compound chosen from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these. According to one or more embodiments, the solvent comprises or consists of sulfolane. According to one or more embodiments, the solvent is constituted of at least 80% by weight (e.g., of at least 90% by weight), preferably of at least 95% by weight (e.g., of at least 99% by weight), of sulfolane, with respect to the total weight of the solvent. According to one or more embodiments, the solvent additionally comprises an anti-solvent, such as water. According to one or more embodiments, the anti-solvent comprises or consists of water. According to one or more embodiments, the solvent comprises between 0.01% by weight and 5% by weight, preferably between 0.1% by weight and 3% by weight (e.g., between 0.5% by weight and 2% by weight), of anti-solvent, such as water, with respect to the total weight of the solvent. According to one or more embodiments, the solvent comprises or consists of sulfolane and water.

According to the invention, a first fraction 51 of extract recycled material 5, for example resulting from the extract stripping section T3, feeds the upper part Z1 of the liquid-liquid extractor, i.e. above the feed point of the feedstock 1. According to one or more embodiments, the first fraction 51 feeds said upper part Z1 preferably below the feed point of the solvent stream 2 in the upper part Z1 of the liquid-liquid extractor T1.

According to one or more embodiments, the first fraction 51 feeds said upper part Z1 at a point arranged at a position of between 0.05×L1 and 0.95×L1, and preferably between 0.2×L1 and 0.9×L1 and preferably between 0.3×L1 and 0.8×L1, L1 being the length (starting) from the top of the liquid-liquid extractor T1 (down) to the position of the feed point of the feedstock 1 corresponding to the length of the upper part Z1. In the present description, 0×L1 corresponds to the position of the top of the liquid-liquid extractor T1. According to one or more embodiments, the first fraction 51 feeds said upper part Z1 at a point arranged at a position of between 0.05×L1 and 0.8×L1, preferably between 0.05×L1 and 0.7×L1, preferably between 0.05×L1 and 0.5×L1 and preferably between 0.05×L1 and 0.6×L1, such as, for example, between 0.1×L1 and 0.6×L1. Advantageously, the high position of the first fraction in the liquid-liquid extractor makes possible a gain with regard to the aromatic extraction yields.

According to the invention, a second fraction 52 of extract recycled material 5 feeds the lower part Z2 of the liquid-liquid extractor T1, i.e. below the feed point of the feedstock 1.

According to one or more embodiments, the second fraction 52 feeds said lower part Z2 at a point arranged at a position of between 0×L2 and 0.3×L2, preferably between 0×L2 and 0.2×L2 and preferably between 0×L2 and 0.1×L2, L2 being the length (starting) from the bottom of the liquid-liquid extractor T1 (up) to the feed point of the feedstock 1 corresponding to the length of the lower part Z2. According to one or more embodiments, the second fraction 52 feeds the bottom of the lower part Z2 of the liquid-liquid extractor T1. In the present description, 0×L2 corresponds to the position of the bottom of the liquid-liquid extractor T1.

According to one or more embodiments, the ratio by weight of the first fraction 51 of the extract recycled material 5 feeding the upper part Z1 to the second fraction 52 of the extract recycled material 5 feeding the lower part Z2 is between 0.01 and 0.99, preferably between 0.1 and 0.95, preferably between 0.15 and 0.85, and preferably between 0.2 and 0.8.

The liquid-liquid extractor T1 makes it possible to separate a raffinate 3 depleted in aromatic compounds with respect to the feedstock 1 and an extract 4 concentrated in aromatic compounds with respect to the feedstock 1. The raffinate 3 exits at the top of the liquid-liquid extractor T1 and is optionally sent to the water scrubbing tower T2. The extract 4 exits at the bottom of the liquid-liquid extractor T1 and is sent to the extract stripping section T3, preferably with exchange of heat with the solvent stream 2 (not represented in FIG. 2 for the sake of simplicity). According to one or more embodiments, the liquid-liquid extractor T1 operates adiabatically.

According to one or more embodiments, the ratio by weight of the solvent 2 to the feedstock 1 is between 0.1 and 50, preferably between 0.5 and 20, preferably between 1 and 9, preferably between 3 and 8, such as 5±1 or 6±1. According to one or more embodiments, the ratio by weight of the extract recycled material 5 to the feedstock 1 is between 0.05 and 10, preferably between 0.1 and 8, preferably between 0.2 and 5, preferably between 0.3 and 2, such as 0.9±0.2.

According to one or more embodiments, the liquid-liquid extractor T1 is operated at a pressure of between 0.05 MPa and 3 MPa (0.5 and 30 bara), preferably between 0.1 MPa and 2 MPa (1 and 20 bara), preferably between 0.2 MPa and 1.5 MPa (2 and 15 bara), preferably between 0.3 MPa and 1 MPa (3 and 10 bara), such as at 0.65±0.2 MPa (6.5+/−2 bara), for example when the solvent comprises sulfolane. According to one or more embodiments, the extractor is operated at a temperature of between 10° C. and 150° C., preferably between 15° C. and 130° C., preferably between 30° C. and 120° C., preferably between 40° C. and 110° C., such as 70±2° C., for example when the solvent comprises sulfolane.

Optional water scrubbing tower T2

The water scrubbing of the raffinate 3 makes possible the removal of the solvent potentially present in the raffinate 3. Preferably, the raffinate 3 is cooled before entering the tower T2. Preferably, the raffinate 3 enters in the bottom of said tower T2, water 6 is introduced at the top of said tower, the non-aromatic stream 7 (scrubbed raffinate 3 thus depleted in solvent) exits at the top of said tower and the aqueous scrubbing liquor 8 (concentrated/enriched in solvent) exits in the bottom of said tower. According to one or more embodiments, the non-aromatic stream 7 comprises less than 100 ppm by weight, preferably less than 10 ppm by weight, very preferably less than 1 ppm by weight, of solvent. According to one or more embodiments, the aqueous scrubbing liquor 8 is sent to the water stripping section (not represented).

Advantageously, the non-aromatic stream 7 comprises less than 25% by weight, preferably less than 20% by weight, very preferably less than 17% by weight, of aromatic compounds, with respect to the total weight of the non-aromatic stream 7.

Advantageously, the non-aromatic stream 7 can be sent to a thermal cracking stage/unit or else to a catalytic reforming stage/unit, for example in order to increase the production of aromatics.

### Extract Stripping Section T3

The stripping of the extract 4 makes possible the removal of non-aromatic compounds still present in the extract 4. The extract 4, charged with aromatic compounds and with solvent, is preferably introduced at the top of the extract stripping section T3. In the extract stripping section T3, the purity of the extract 4 is improved as the residual non-aromatic compounds entrained with the aromatic compounds (less soluble in the solvent) are extracted in the form of a gas stream 9 at the top of the extract stripping section T3. According to one or more embodiments, the gas stream 9 is combined with water, such as the water recovered at the top of the water stripping section (not represented). Phase separation in the condenser-settler CD3 makes possible the recycling of the hydrocarbon phase to the liquid-liquid extractor T1 as stream of extract recycled material 5 and the aqueous phase 10 is optionally combined with the aqueous scrubbing liquor 8 and sent to the water stripping section (not represented). The purified extract 12 resulting from the extract stripping section T3 can be sent to the aromatic recovery tower T6 in order to separate the aromatics from the solvent (in particular from sulfolane), which can be recycled to the liquid-liquid extractor T1.

According to one or more embodiments, the extract recycled material comprises between 50% and 95% by weight, preferably between 60% and 90% by weight (e.g. 70-90% by weight), of non-aromatic compounds (compounds entrained by the aromatic compounds of the extract). According to one or more embodiments, the extract recycled material comprises at least 70% by weight, preferably at least 80% by weight, very preferably at least 90% by weight, of compounds having 7 or fewer carbon atoms.

The stripping of the extract 4 is preferably carried out at low pressure or even under vacuum in order to improve the removal of the non-aromatics from the solvent (in particular from sulfolane). According to one or more embodiments, the extract stripping section T3 is operated at a top pressure of between 0.001 MPa and 2 MPa (0.01 and 20 bara), preferably between 0.005 MPa and 1 MPa (0.05 and 10 bara), preferably between 0.01 MPa and 0.8 MPa (0.1 and 8 bara), preferably between 0.03 MPa and 0.5 MPa (0.3 and 5 bara), such as 0.1±0.05 MPa, for example when the solvent comprises sulfolane. According to one or more embodiments, the extract stripping section T3 is operated at a bottom temperature of between 50° C. and 300° C., preferably between 100° C. and 250° C., preferably between 130° C. and 200° C., preferably between 145° C. and 195° C., such as 180±5° C., for example when the solvent comprises sulfolane.

### Water Stripping Section (Optional—not Represented)

The stripping of the aqueous scrubbing liquor 8 and possibly of the aqueous phase 10 makes it possible to remove the dissolved hydrocarbons (mainly non-aromatics) from the water resulting from the top of the extract stripping section T3 and optionally from the water scrubbing tower T2.

According to one or more embodiments, at least a part of the water obtained at the outlet of the water stripping section is sent into the aromatic recovery tower T6 in order to increase the amount of stripping steam sent into the bottom of the aromatic recovery tower T6.

### Aromatic Recovery Tower T6

The aromatic recovery tower T6 separates the purified extract 12 resulting from the bottom of the extract stripping section T3 into an aromatic stream 13 and the solvent stream 2. Preferably, the aromatic recovery tower T6 operates under vacuum, in particular when the solvent comprises sulfolane. Advantageously, the operation under vacuum makes it possible to avoid an excessive bottom temperature, which might result in the decomposition of the solvent. According to one or more embodiments, steam 11, optionally combined with solvent (regenerated), is injected into the bottom of the aromatic recovery tower T6, in order to improve the extraction of the aromatics from the solvent (in particular from sulfolane). The top vapours from the tower (comprising essentially aromatic compounds and optionally water) can be condensed and optionally separated by settling in a condenser-settler CD6. According to one or more embodiments, a part of the condensed top aromatic vapours is used for the reflux of the aromatic recovery tower T6 and the remainder, constituting the aromatic stream 13, exits from the process and is preferably sent to an aromatics complex (unit for the separation and production of aromatic compounds and in particular of BTX compounds). A part of the water 14 separated by settling in the condenser-settler CD6 can optionally be sent to the water scrubbing tower T2 and the remainder is recycled to the water stripping section. The stream resulting from the bottom of the aromatic recovery tower T6 is composed of regenerated solvent which can be sent to the liquid-liquid extractor T1, and optionally to the extract stripping section T3 and/or to the solvent regeneration section (not represented).

According to one or more embodiments, the aromatic recovery tower T6 is operated at a top pressure of between 0.001 MPa and 2 MPa (0.01 and 20 bara), preferably between 0.005 MPa and 1 MPa (0.05 and 10 bara), preferably between 0.01 MPa and 0.5 MPa (0.1 and 5 bara), preferably between 0.015 MPa and 0.2 MPa (0.15 and 2 bara), such as 0.06±0.04 MPa, for example when the solvent comprises sulfolane. According to one or more embodiments, the aromatic recovery tower T6 is operated at a bottom temperature of between 50° C. and 300° C., preferably between 100° C. and 250° C., preferably between 120° C. and 200° C., preferably between 130° C. and 195° C., such as 170±25° C., for example when the solvent comprises sulfolane.

Advantageously, the aromatic stream 13 comprises at least 95% by weight, preferably at least 99% by weight, very preferably at least 99.5% by weight (e.g., at least 99.5% by weight), of aromatic compounds, with respect to the total weight of the aromatic stream 13.

### EXAMPLES

### Example 1: Comparative Example (not in Accordance with the Invention)

A comparative example of process for the separation of the aromatics originating from a petrol cut resulting from catalytic cracking technologies is as follows.

The characteristics of the feedstock 1 treated in the separation process are shown in Table 1.

TABLE 1

| Content | Unit | Feedstock 1 |
|---|---|---|
| Paraffins | % by weight | 32.8 |
| Olefins | % by weight | 0.0 |
| Naphthenes | % by weight | 12.9 |
| Aromatics | % by weight | 54.3 |
| including C6-C7 aromatics | % by weight | 16.6 |
| including C8-C9 aromatics | % by weight | 26.8 |
| including C10 aromatics | % by weight | 8.8 |
| including C11+ aromatics | % by weight | 2.1 |

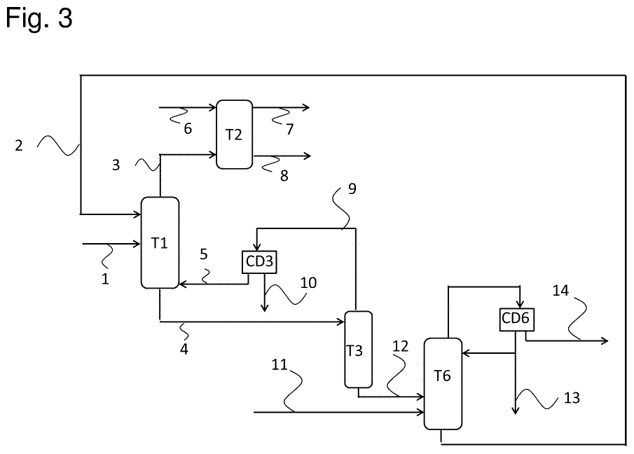

The feedstock 1 feeds the liquid-liquid extractor T1 of the aromatic extraction unit. A solvent stream 2 comprising 99.1% by weight of sulfolane is added at the top and a stream of extract recycled material 5 is added at the bottom of the liquid-liquid extractor T1, as shown in FIG. 3. A [solvent stream 2]/[feedstock 1] ratio by weight of 8 and a [stream of extract recycled material 5]/[feedstock 1] ratio by weight of 0.5 are involved at the liquid-liquid extractor T1, which is operated at a pressure between 0.4 MPa and 0.8 MPa and at a temperature of 70° C.

The raffinate 3 is extracted at the top of the liquid-liquid extractor T1 and then sent to the optional water scrubbing tower T2 in order to remove the small amount of sulfolane solvent which is entrained in the raffinate 3. The aqueous scrubbing liquor 8 is composed essentially of water and sulfolane, and the non-aromatic stream 7 is composed of the raffinate devoid of solvent.

For the sake of simplicity, the optional water stripping and solvent regeneration sections are not represented in FIG. 3. Said optional water stripping and solvent regeneration sections make it possible to treat the water streams used in the separation process and to purify the sulfolane.

The extract 4 from the liquid-liquid extractor T1 feeds the extract stripping section T3, which makes it possible to remove the non-aromatics entrained with the solvent and the aromatics and to create the stream of extract recycled material 5 satisfactory for returning to the liquid-liquid extractor T1. Beforehand, the gas stream 9 at the top of the extract stripping section T3 is sent to the condenser-settler CD3 in order to remove the aqueous phase 10.

The extract stripping section T3 comprises a stripping column operated at a bottom temperature of 180° C.

The purified extract 12 at the bottom of the extract stripping section T3, containing essentially the solvent and aromatics, is sent to the aromatic recovery tower T6, which makes it possible to recover, at the bottom, the solvent recycled to the liquid-liquid extractor T1 as solvent stream 2 and to recover a top stream which is sent to the condenser-settler CD6, which makes it possible to withdraw a water 14 separated by settling and an aromatic stream 13.

The aromatic recovery tower T6 operates by sending steam 11 into the column bottom.

The aromatic recovery tower T6 is operated at a bottom temperature of 180° C.

The compositions by weight of the exiting streams of the reference separation process are presented in Table 2.

TABLE 2

| | | Non-aromatic | Aromatic |
|---|---|---|---|
| Content | Unit | stream 7 | stream 13 |
| Paraffins | % by weight | 67.7 | 0.0 |
| Olefins | % by weight | 0.0 | 0.0 |
| Naphthenes | % by weight | 26.0 | 0.1 |
| Aromatics | % by weight | 6.1 | 99.7 |
| including C6-C7 aromatics | % by weight | 0.0 | 32.2 |
| including C8-C9 aromatics | % by weight | 1.3 | 50.9 |
| including C10 aromatics | % by weight | 2.6 | 14.6 |
| including C11+ aromatics | % by weight | 2.2 | 2.0 |
| Water | % by weight | 0.2 | 0.2 |

The distribution by weight of the streams of the reference separation process at the inlet and outlet of the aromatic extraction unit is presented in Table 3.

TABLE 3

| | Distribution of the feedstock |
|---|---|
| Stream | (fraction by weight) |
| Feedstock 1 | 1 |
| Non-aromatic stream 7 | 0.48 |
| Aromatic stream 13 | 0.52 |

The extraction yields of the aromatic compounds in the aromatic stream 13 with respect to the feedstock 1 are presented in Table 4.

TABLE 4

| Extraction | % by |
|---|---|
| yield | weight |
| A6 | 99.9 |
| A7 | 99.9 |
| A8 | 99.7 |
| A9 | 95.5 |
| A10 | 85.5 |
| A11 | 48.7 |
| Total | 94.5 |

### Example 2 According to the Invention

An example of separation process according to the invention for the separation of the aromatics originating from a petrol cut resulting from catalytic cracking technologies is as follows.

This example describes the technical effect of the introduction of two fractions of the stream of extract recycled material 5 corresponding to the first fraction 51 and the second fraction 52 according to FIG. 1, into the upper part Z1 and the lower part Z2 of the liquid-liquid extractor T1, respectively.

The feedstock 1 (which is identical to the feedstock of Example 1) feeds the liquid-liquid extractor T1 of the aromatic extraction unit. A solvent stream 2 comprising 99.1% by weight of sulfolane is added at the top. The stream 52, corresponding to 50% by weight of the stream of extract recycled material 5, is added at the bottom of the liquid-liquid extractor T1 in the lower part Z2, as shown in FIGS. 1 and 2. The stream 51, corresponding to 50% by weight of the stream of extract recycled material 5, is added in the upper part Z1 of the liquid-liquid extractor T1 in the 0.66×L1 position, 0×L1 corresponding to the top of the upper part Z1.

A [solvent stream 2]/[feedstock 1] ratio by weight of 8 and a [stream of extract recycled material 5]/[feedstock 1] ratio by weight of 0.28 are involved at the liquid-liquid extractor T1, which is operated at a pressure between 0.4 MPa and 0.8 MPa and at a temperature of 70° C.

The raffinate 3 is extracted at the top of the liquid-liquid extractor T1 and then sent to the optional water scrubbing tower T2 in order to remove the small amount of sulfolane solvent which is entrained in the raffinate 3. The aqueous scrubbing liquor 8 is composed essentially of water and sulfolane, and the non-aromatic stream 7 is composed of the raffinate devoid of solvent.

For the sake of simplicity, the optional water stripping and solvent regeneration sections are not represented in FIG. 1. Said optional water stripping and solvent regeneration sections make it possible to treat the water streams used in the separation process according to the invention and to purify the sulfolane.

The extract 4 from the liquid-liquid extractor T1 feeds the extract stripping section T3, which makes it possible to remove the non-aromatics entrained with the solvent and the aromatics and to create the stream of extract recycled material 5 satisfactory for returning to the liquid-liquid extractor T1. Beforehand, the gas stream 9 at the top of the extract stripping section T3 is sent to the condenser-settler CD3 in order to remove the aqueous phase 10.

The extract stripping section T3 comprises a stripping column operated at a bottom temperature of 180° C.

The purified extract 12 at the bottom of the extract stripping section T3, containing essentially the solvent and aromatics, is sent to the aromatic recovery tower T6, which makes it possible to recover, at the bottom, the solvent recycled to the liquid-liquid extractor T1 as solvent stream 2 and to recover a top stream which is sent to the condenser-settler CD6, which makes it possible to withdraw a water 14 separated by settling and an aromatic stream 13.

The aromatic recovery tower T6 operates by sending steam 11 into the column bottom.

The aromatic recovery tower T6 is operated at a bottom temperature of 180° C.

The compositions by weight of the exiting streams of the separation process according to the invention are presented in Table 5.

TABLE 5

| | | Non-aromatic | Aromatic |
|---|---|---|---|
| Content | Unit | stream 7 | stream 13 |
| Paraffins | % by weight | 69.4 | 0.2 |
| Olefins | % by weight | 0.0 | 0.0 |
| Naphthenes | % by weight | 26.7 | 0.6 |
| Aromatics | % by weight | 3.7 | 99.0 |
| including C6-C7 aromatics | % by weight | 0.0 | 31.2 |
| including C8-C9 aromatics | % by weight | 0.7 | 49.9 |
| including C10 aromatics | % by weight | 1.8 | 15.0 |
| including C11+ aromatics | % by weight | 1.2 | 2.9 |
| Water | % by weight | 0.2 | 0.2 |

The distribution by weight of the streams of the separation process at the inlet and outlet of the aromatic extraction unit is presented in Table 6.

TABLE 6

| | Distribution of the feedstock |
|---|---|
| Stream | (fraction by weight) |
| Feedstock 1 | 1 |
| Non-aromatic stream 7 | 0.47 |
| Aromatic stream 13 | 0.53 |

The extraction yields of the aromatic compounds in the aromatic stream 13 with respect to the feedstock 1 are presented in Table 7.

TABLE 7

| Extraction | % by |
|---|---|
| yield | weight |
| A6 | 99.9 |
| A7 | 99.9 |
| A8 | 99.8 |
| A9 | 97.6 |
| A10 | 90.6 |
| A11 | 72.9 |
| Total | 96.8 |

Example 2 according to the invention demonstrates an improvement in the extraction yield of the aromatics with respect to Reference Example 1.

### Example 3 According to the Invention

An example of separation process according to the invention for the separation of the aromatics originating from a petrol cut resulting from catalytic cracking technologies is as follows.

The feedstock 1 (which is identical to the feedstock of Example 1) feeds the liquid-liquid extractor T1 of the aromatic extraction unit. A solvent stream 2 comprising 99.1% by weight of sulfolane is added at the top. The stream 52, corresponding to 50% by weight of the stream of extract recycled material 5, is added at the bottom of the liquid-liquid extractor T1 in the lower part Z2, as shown in FIGS. 1 and 2. The stream 51, corresponding to 50% by weight of the stream of extract recycled material 5, is added in the upper part Z1 of the liquid-liquid extractor T1 in the 0.33×L1 position, 0×L1 corresponding to the top of the upper part Z1.

A [solvent stream 2]/[feedstock 1] ratio by weight of 8 and a [stream of extract recycled material 5]/[feedstock 1] ratio by weight of 0.27 are involved at the liquid-liquid extractor T1, which is operated at a pressure between 0.4 MPa and 0.8 MPa and at a temperature of 70° C.

The raffinate 3 is extracted at the top of the liquid-liquid extractor T1 and then sent to the optional water scrubbing tower T2 in order to remove the small amount of sulfolane solvent which is entrained in the raffinate 3. The aqueous scrubbing liquor 8 is composed essentially of water and sulfolane, and the non-aromatic stream 7 is composed of the raffinate devoid of solvent.

For the sake of simplicity, the optional water stripping and solvent regeneration sections are not represented in FIG. 1. Said optional water stripping and solvent regeneration sections make it possible to treat the water streams used in the separation process according to the invention and to purify the sulfolane.

The extract 4 from the liquid-liquid extractor T1 feeds the extract stripping section T3, which makes it possible to remove the non-aromatics entrained with the solvent and the aromatics and to create the stream of extract recycled material 5 satisfactory for returning to the liquid-liquid extractor T1. Beforehand, the gas stream 9 at the top of the extract stripping section T3 is sent to the condenser-settler CD3 in order to remove the aqueous phase 10.

The extract stripping section T3 comprises a stripping column operated at a bottom temperature of 180° C.

The purified extract 12 at the bottom of the extract stripping section T3, containing essentially the solvent and aromatics, is sent to the aromatic recovery tower T6, which makes it possible to recover, at the bottom, the solvent recycled to the liquid-liquid extractor T1 as solvent stream 2 and to recover a top stream which is sent to the condenser-settler CD6, which makes it possible to withdraw a water 14 separated by settling and an aromatic stream 13.

The aromatic recovery tower T6 operates by sending steam 11 into the column bottom.

The aromatic recovery tower T6 is operated at a bottom temperature of 180° C.

The compositions by weight of the exiting streams of the separation process according to the invention are presented in Table 8.

TABLE 8

| | | Non-aromatic | Aromatic |
|---|---|---|---|
| Content | Unit | stream 7 | stream 13 |
| Paraffins | % by weight | 69.9 | 0.3 |
| Olefins | % by weight | 0.0 | 0.0 |
| Naphthenes | % by weight | 26.9 | 0.6 |
| Aromatics | % by weight | 3.0 | 98.9 |
| including C6-C7 aromatics | % by weight | 0.1 | 31.0 |
| including C8-C9 aromatics | % by weight | 0.5 | 49.7 |
| including C10 aromatics | % by weight | 1.4 | 15.2 |
| including C11+ aromatics | % by weight | 1.0 | 3.0 |
| Water | % by weight | 0.2 | 0.2 |

The distribution by weight of the streams of the separation process between the inlet and the outlet of the aromatic extraction unit is presented in Table 9.

TABLE 9

| | Distribution of the feedstock |
|---|---|
| Stream | (fraction by weight) |
| Feedstock 1 | 1 |
| Non-aromatic stream 7 | 0.47 |
| Aromatic stream 13 | 0.53 |

The extraction yields of the aromatic compounds in the aromatic stream 13 with respect to the feedstock 1 are presented in Table 10.

TABLE 10

| Extraction | % by |
|---|---|
| yield | weight |
| A6 | 99.8 |
| A7 | 99.9 |
| A8 | 99.8 |
| A9 | 98.2 |
| A10 | 92.3 |
| A11 | 77.5 |
| Total | 97.3 |

Example 3 according to the invention demonstrates an improvement in the extraction yield of the total aromatics with respect to Reference Example 1. In addition, Example 3 according to the invention also demonstrates an improvement for the recovery of the heaviest aromatics with respect to Example 2 according to the invention (see Table 7 vs Table 10).

1. Process for the separation of aromatic compounds from a feedstock (1) comprising a mixture of aromatic and non-aromatic compounds, the process comprising the following stages:
extracting the aromatics from the feedstock (1) by means of a liquid-liquid extractor (T1) fed with a solvent stream (2), in order to produce at least an extract (4) concentrated in aromatic compounds, with respect to the composition of the feedstock (1), and a raffinate (3) concentrated in non-aromatic compounds, with respect to the composition of the feedstock (1);
feeding an upper part (Z1) and a lower part (Z2) of the liquid-liquid extractor (T1) with respectively a first fraction (51) and a second fraction (52) of extract recycled material (5), the upper part (Z1) and the lower part (Z2) being defined with respect to the position of the feed point of the feedstock (1) in the liquid-liquid extractor (T1).

2. Process according to claim 1, in which the solvent stream 2 feeds the top of the upper part Z1 of the liquid-liquid extractor T1.

3. Process according to claim 1, in which the first fraction (51) feeds said upper part (Z1) at a point arranged at a position of between 0.05×L1 and 0.95×L1, preferably between 0.2×L1 and 0.9×L1 and preferably between 0.3×L1 and 0.8×L1, L1 being the length from the top of the liquid-liquid extractor T1 to the position of the feed point of the feedstock (1).

4. Process according to a claim 1, in which the first fraction (51) feeds said upper part (Z1) below the feed point of the solvent stream (2).

5. Process according to claim 1, in which the second fraction (52) feeds said lower part (Z2) at a point arranged at a position of between 0×L2 and 0.3×L2, preferably between 0×L2 and 0.2×L2 and preferably between 0×L2 and 0.1×L2, L2 being the length from the position of the bottom of the liquid-liquid extractor T1 to the feed point of the feedstock (1).

6. Process according to claim 1, in which the ratio by weight of the first fraction (51) to the second fraction (52) is of between 0.01 and 0.99, preferably between 0.1 and 0.95, preferably between 0.15 and 0.85 and preferably between 0.2 and 0.8.

7. Process according to claim 1, in which the ratio by weight of the solvent (2) to the feedstock (1) is of between 0.1 and 50, preferably between 0.5 and 20, preferably between 1 and 9, preferably between 3 and 8.

8. Process according to claim 1, in which the ratio by weight of the extract recycled material (5) to the feedstock (1) is of between 0.05 and 10, preferably between 0.1 and 8, preferably between 0.2 and 5, preferably between 0.3 and 2.

9. Process according to claim 1, in which the feedstock (1) contains aromatic and non-aromatic compounds comprising from 6 to 11 carbon atoms.

10. Process according to claim 1, in which the liquid-liquid extractor (T1) is operated at at least one of the following operating conditions: a pressure of between 0.05 MPa and 3 MPa, preferably between 0.1 MPa and 2 MPa, preferably between 0.2 MPa and 1.5 MPa, preferably between 0.3 MPa and 1 MPa; a temperature of between 10° C. and 150° C., preferably between 15° C. and 130° C., preferably between 30° C. and 120° C., preferably between 40° C. and 110° C.

11. Process according to claim 1, comprising the following stages:
stripping of the extract (4) by means of an extract stripping section (T3) in order to separate a gas stream (9) comprising non-aromatic compounds and a purified extract (12);
separation of the aromatics from the purified extract (12) by means of an aromatic recovery tower (T6) in order to separate the solvent stream (2) and top vapours comprising an aromatic stream (13).

12. Process according to claim 11, comprising at least one of the following stages:
feeding the liquid-liquid extractor (T1) with a solvent stream (2) comprising a solvent chosen from the list consisting of ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these;
phase separation of the gas stream (9) in a condenser-settler (CD3) in order to separate the extract recycled material (5) and an aqueous phase (10);
scrubbing the raffinate (3) with water by means of a water scrubbing tower (T2) fed with water (6) in order to produce a non-aromatic stream (7) and aqueous scrubbing liquor (8);
regeneration of at least a portion of the solvent stream (2) by means of a section for regeneration of the solvent by vacuum steam stripping;
condensation of the top vapours in a condenser-settler (CD6) in order to produce the aromatic stream (13).

13. Process according to claim 12, comprising at least one of the following stages:
feeding the liquid-liquid extractor (T1) with a stream of solvent chosen from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these;
stripping the hydrocarbon compounds present in the aqueous scrubbing liquor (8) and/or in the aqueous phase (10) by means of a water stripping section in order to produce water;
feeding the aromatic recovery tower (T6) with steam (11) in order to produce top vapours additionally comprising steam, and condensation of the top vapours in the condenser-settler (CD6) in order to produce the aromatic stream (13) and water (14) separated by settling.

14. Process according to claim 13, comprising at least one of the following stages:
feeding the liquid-liquid extractor (T1) with a solvent stream (2) comprising sulfolane and water;
recycling the water produced by the water stripping section to the aromatic recovery tower (T6).

15. Device for the separation of aromatic compounds from a feedstock (1) comprising a mixture of aromatic and non-aromatic compounds, the device comprising a liquid-liquid extractor (T1) suitable for extracting aromatics from the feedstock (1) with a solvent stream (2) and producing at least an extract (4) concentrated in aromatic compounds, with respect to the feedstock (1), and a raffinate (3) concentrated in non-aromatic compounds, with respect to the composition of the feedstock (1), the liquid-liquid extractor (T1) comprising:
a feed point for the feedstock (1);
a feed point for the solvent stream (2);
a feed point for a first fraction (51) of extract recycled material (5) arranged in an upper part (Z1) of the liquid-liquid extractor (T1);
a feed point for a second fraction (52) of extract recycled material (5) arranged in a lower part (Z2) of the liquid-liquid extractor (T1), the upper part (Z1) and the lower part (Z2) being defined with respect to the position of the feed point of the feedstock (1) in the liquid-liquid extractor (T1).
